(19) United States
(12) Patent Application Publication
(10) Pub. No.: US 2024/0003904 A1
(43) Pub. Date: Jan. 4, 2024

(54) BIOMARKER-BASED RISK MODEL TO PREDICT PERSISTENT MULTIPLE ORGAN DYSFUNCTION AFTER CONGENITAL HEART SURGERY

(71) Applicant: Children's Hospital Medical Center, Cincinnati, OH (US)

(72) Inventor: Hector R. Wong, Cincinnati, OH (US)

(73) Assignee: Children's Hospital Medical Center, Cincinnati, OH (US)

(21) Appl. No.: 18/325,890

(22) Filed: May 30, 2023

Related U.S. Application Data
(60) Provisional application No. 63/347,504, filed on May 31, 2022.

(51) Int. Cl. G01N 33/68

(52) U.S. Cl. CPC G01N 33/6869; G01N 2800/56

(57) ABSTRACT

Methods and compositions disclosed herein generally relate to methods of identifying, validating, and measuring clinically relevant, quantifiable biomarkers of diagnostic and therapeutic responses for blood, vascular, cardiac, and respiratory tract dysfunction, particularly as those responses relate to persistent multiple organ dysfunction syndrome (MODS) in pediatric patients following cardiopulmonary bypass (CPB). Certain aspects of the disclosure relates to identifying one or more biomarkers associated with septic shock in pediatric patients, obtaining one or more samples from a pediatric patient following CPB, then quantifying from the sample an amount of said biomarkers, wherein the level of said biomarker correlates with a predicted outcome.

### CROSS REFERENCE TO RELATED APPLICATION

The present application claims the benefit of priority under 35 U.S.C. § 119(e) to U.S. Provisional Application No. 63/347,504, PREDICTING PERSISTENT MULTIPLE ORGAN DYSFUNCTION IN THE PEDIATRIC POPULATION AFTER CARDIOPULMONARY BYPASS USING SEPSIS PROGNOSTIC BIOMARKERS, filed on May 31, 2022, which is currently co-pending herewith and which is incorporated by reference in its entirety.

### STATEMENT REGARDING FEDERALLY-SPONSORED RESEARCH

This invention was made with government support under R35GM126943 awarded by the National Institutes of Health. The government has certain rights in the invention.

### FIELD

The disclosure herein generally relates to the identification, validation, and applications of clinically relevant, quantifiable biomarkers associated with sepsis and septic shock, and in more particular aspects to pediatric patients at risk of developing multiple organ dysfunction following cardiopulmonary bypass.

### BACKGROUND

Multiple organ dysfunction syndrome (MODS) is an important cause of post-operative morbidity and mortality for children undergoing cardiac surgery requiring cardiopulmonary bypass (CPB). Dysregulated inflammation is widely regarded as a key contributor to bypass-related MODS pathobiology, with considerable overlap of pathways associated with septic shock.

Cardiopulmonary bypass CPB potentiates a systemic inflammatory response in all patients, the degree of which varies based on many factors [1-9]. An exaggerated response, as seen in systemic inflammatory response syndrome (SIRS), can be detrimental and contributes to the development of MODS, prolonged length of stay, and worse outcomes [5-7].

Almost all pediatric cardiac surgery patients meet criteria for organ dysfunction in the early postoperative period with ubiquitous inotropic and/or mechanical ventilator support, but children with optimal surgical interventions will begin to wean from postoperative support within the first few days. Failure to wean may represent persistent or progressive organ dysfunction, with risk of mortality increasing in conjunction with number of organ systems involved [10, 11].

Thus, there is an urgent need to identify patients at increased risk for persistent MODS due to an exaggerated inflammatory response to CPB. Such patient stratification can help guide clinical management, provide prognostic enrichment in future trials, and, ultimately, improve outcomes.

### SUMMARY

Embodiments of the disclosure include methods of classifying a patient following cardiopulmonary bypass (CPB) as high risk of persistent multiple organ dysfunction syndrome (MODS), or other than high risk of persistent MODS, the methods including: obtaining a sample from a pediatric patient at about 12 hours post-CPB; analyzing the 12 hours post-CPB sample to determine expression levels of one or more biomarkers comprising IL-8; determining whether the expression level of IL-8 at 12 hours is greater than a respective cut-off IL-8 expression level; and classifying the patient as high risk of persistent MODS, or other than high risk of persistent MODS, based on the determination of whether the expression level of IL-8 at 12 hours is greater than the respective cut-off IL-8 expression level.

In some embodiments, the methods further include: determining whether the patient age is greater than 12 months; and classifying the patient as high risk of persistent MODS, or other than high risk of persistent MODS, based on the determination of whether the expression level of IL-8 at 12 hours is greater than the respective cut-off IL-8 expression level, and whether the patient age is greater than 12 months.

In some embodiments, the methods further include: obtaining a sample from a pediatric patient at about 4 hours post-CPB; analyzing the 4 hours post-CPB sample to determine expression levels of one or more biomarkers comprising CCL3; analyzing the 12 hours post-CPB sample to determine expression levels of one or more biomarkers comprising CCL3; determining whether the change in expression level of CCL-3 from 4 to 12 hours is greater than a respective cut-off delta; and classifying the patient as high risk of persistent MODS, or other than high risk of persistent MODS, based on the determination of whether the expression level of IL-8 at 12 hours is greater than the respective cut-off IL-8 expression level, whether the change in expression level of CCL-3 from 4 to 12 hours is greater than a respective cut-off delta, and whether the patient age is greater than 12 months.

In some embodiments, a classification of high risk of persistent MODS includes: a) an elevated level of IL-8; and a classification of other than high risk of persistent MODS includes: b) a non-elevated level of IL-8, and a patient age greater than 12 months; or c) a non-elevated level of IL-8, and a patient age of less than or equal to 12 months.

In some embodiments, a classification other than high risk includes a classification of low risk or intermediate risk. In some embodiments, a classification of intermediate risk of persistent MODS includes: a non-elevated level of IL-8, a patient age of less than or equal to 12 months, and a non-elevated CCL3 delta; and a classification of low risk of persistent MODS includes: a non-elevated level of IL-8, and a patient age of less than or equal to 12 months, and an elevated CCL3 delta; or a non-elevated level of IL-8, and a patient age greater than 12 months.

In some embodiments, biomarker expression levels can be determined by quantification of serum protein biomarker concentrations. In some embodiments, biomarker expression levels can be determined by concentrations and/or by cycle threshold (CT) values. In some embodiments, the determined biomarker expression levels include expression levels of IL-8 and CCL3.

In some embodiments, biomarker levels can be determined by serum protein biomarker concentration, and: a) an elevated level of IL-8 corresponds to a serum IL-8 concentration greater than 125 pg/ml; and b) an elevated CCL3 delta corresponds to a CCL3 delta greater than −6 pg/ml.

In some embodiments, the determination of whether the levels of the at least two biomarkers are non-elevated above a cut-off level includes applying the biomarker expression level data to a decision tree comprising the two or more biomarkers. In some embodiments, the biomarker expression level data can be applied to the decision tree of FIG. 2.

In some embodiments, persistent MODS includes cardiovascular, respiratory, renal, hepatic, hematologic, and/or neurologic dysfunction, and/or systemic inflammation. In some embodiments, persistent MODS includes renal dysfunction, and/or increase in days requiring mechanical ventilatory support and cardiovascular support (e.g. use of vasoactive-inotropic infusion). In some embodiments, the can be is undergoing continuous renal replacement therapy (CRRT).

In some embodiments, the classification can be combined with one or more patient demographic data and/or clinical characteristics and/or results from other tests or indicia of organ dysfunction and/or one or more additional biomarkers and/or platelet count. In some embodiments, the one or more additional biomarkers can be selected from: heat shock protein 70 kDa 1B (HSP70, HSPA1B), C—C Chemokine ligand 4 (CCL4), Granzyme B (GZMB), Interleukin-1α (IL-1α), Matrix metallopeptidase 8 (MMP8), Angiopoietin-1 (Angpt-1), Inter-Cellular Adhesion Molecule-1 (ICAM-1), Vascular cell adhesion molecule-1 (VCAM-1), P-selectin, E-selectin, and Platelet and endothelial cell adhesion molecule-1 (PECAM-1). In some embodiments, the one or more additional biomarkers can be selected from: GZMB, HSP70, IL-1α, CCL4, and MMP8. In some embodiments, the patient demographic data and/or clinical characteristics and/or results from other tests or indicia of organ dysfunction include at least one selected from: the presence or absence or chronic disease, and/or the gender, race, ethnicity, and/or co-morbidities of the patient.

In some embodiments, the classification can be combined with one or more additional population-based risk scores. In some embodiments, the one or more population-based risk scores includes at least one selected from: Pediatric Sepsis Biomarker Risk Model (PERSEVERE), Pediatric Sepsis Biomarker Risk Model II (PERSEVERE II), Pediatric Risk of Mortality (PRISM), PRISM III, Pediatric Index of Mortality (PIM), and Pediatric Logistic Organ Dysfunction (PELOD). In some embodiments, the one or more population-based risk scores includes PERSEVERE or PERSEVERE II.

Some embodiments of the methods further include administering a treatment including one or more high risk therapy to a patient that is classified as high risk, or administering a treatment excluding a high risk therapy to a patient that is not high risk, or to provide a method of treating a pediatric patient following CPB. In some embodiments, the one or more high risk therapy includes at least one selected from: biological and/or immune enhancing therapy, extracorporeal membrane oxygenation/life support, plasmapheresis, peritoneal dialysis, pulmonary artery catheterization, high volume continuous hemofiltration, steroids, adjuvant hemoperfusion, and/or plasma filtration and/or adsorption therapies. In some embodiments, the biological and/or immune enhancing therapy includes administration of GM-CSF, Interleukin-1 receptor antagonist, Interleukin-7, and/or anti-PD-1.

In some embodiments, the patient can be enrolled in a clinical trial. In some embodiments, the patient enrolled in a clinical trial can be classified as high risk. In some embodiments, the methods include prognostic enrichment through enrollment of the high risk patient in the clinical trial. Some embodiments of the methods further include administering a treatment comprising one or more high risk therapy to the patient in the clinical trial.

In some embodiments, the risk of persistent MODS includes a risk of developing persistent MODS by day 5 following CPB. In some embodiments, the methods further include improving an outcome in a pediatric patient following CPB.

In some embodiments, the methods can be part of a companion diagnostic or a point of care device or kit.

Embodiments of the disclosure also include diagnostic kits, tests, or arrays including a reporter hybridization probe, and a capture hybridization probe specific for each of two or more mRNA, DNA, or protein biomarkers selected from: IL-8 and CCL3. In some embodiments, the biomarkers further include one or more of heat shock protein 70 kDa 1B (HSP70, HSPA1B), C—C Chemokine ligand 4 (CCL4), Granzyme B (GZMB), Interleukin-1α (IL-1a), and/or Matrix metallopeptidase 8 (MMP8). In some embodiments, the diagnostic kits, tests, or arrays further include a collection cartridge for immobilization of the hybridization probes. In some embodiments, the reporter and the capture hybridization probes include signal and barcode elements, respectively.

Embodiments of the disclosure also include apparatuses or processing devices suitable for detecting two or more biomarkers selected from: IL-8 and CCL3. In some embodiments, the biomarkers further include one or more of heat shock protein 70 kDa 1B (HSP70, HSPA1B), C—C Chemokine ligand 4 (CCL4), Granzyme B (GZMB), Interleukin-1α (IL-1α), and/or Matrix metallopeptidase 8 (MMP8).

Embodiments of the disclosure also include compositions including a reporter hybridization probe, and a capture hybridization probe specific for each of two or more biomarkers selected from: IL-8 and CCL3. In some embodiments, the biomarkers further include one or more of heat shock protein 70 kDa 1B (HSP70, HSPA1B), C—C Chemokine ligand 4 (CCL4), Granzyme B (GZMB), Interleukin-1α (IL-1α), and/or Matrix metallopeptidase 8 (MMP8).

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 illustrates a comparison of biomarker concentrations in patients with and without persistent MODS.

FIG. 2 illustrates an exemplary derivation classification tree for PERSEVERE-CPB model, which includes the 12-hour interleukin-8 (IL8) serum concentration, the change in C—C chemokine ligand 3 (CCL3) serum concentration from 4 to 12 hours, and the child's age. Terminal nodes 1 and 3 were considered low-risk nodes, with subjects being less likely to develop persistent MODS, while terminal nodes 2 and 4 were considered high-risk and more predictive of development of persistent MODS.

FIG. 3 illustrates an exemplary comparison of PERSEVERE-CPB to validated risk-assessment tools to predict persistent MODS, showing that PERSEVERE-CPB functioned well as a predictor of multiple organ dysfunction syndrome, with cross-validation area under the curve (AUC) that was comparable to validated risk-assessment tools in the cohort. (PERSEVERE: PERSEVERE-CPB biomarker prediction model; STAT: Society of Thoracic Surgery-European Association for Cardiothoracic Surgery mortality category; PRISM-III (pre): Pediatric Risk of Mortality score calculated using preoperative data; PRISM-III (post): Pediatric Risk of Mortality score calculated using data from the first 24 hours after surgery; PELOD-2: Pediatric Logistic Organ Dysfunction Score-2.)

### DETAILED DESCRIPTION

All references cited herein are incorporated by reference in their entirety. Also incorporated herein by reference in their entirety include: U.S. Patent Application No. 61/595,996, BIOMARKERS OF SEPTIC SHOCK, filed on Feb. 7, 2012; U.S. Provisional Application No. 61/721,705, A MULTI-BIOMARKER-BASED OUTCOME RISK STRATIFICATION MODEL FOR ADULT SEPTIC SHOCK, filed on Nov. 2, 2012; International Patent Application No. PCT/US13/25223, A MULTI-BIOMARKER-BASED OUTCOME RISK STRATIFICATION MODEL FOR PEDIATRIC SEPTIC SHOCK, filed on Feb. 7, 2013; International Patent Application No. PCT/US13/25221, A MULTI-BIOMARKER-BASED OUTCOME RISK STRATIFICATION MODEL FOR ADULT SEPTIC SHOCK, filed on Feb. 7, 2013; U.S. Provisional Application No. 61/908,613, TEMPORAL PEDIATRIC SEPSIS BIOMARKER RISK MODEL, filed on Nov. 25, 2013; International Patent Application No. PCT/US14/067438, TEMPORAL PEDIATRIC SEPSIS BIOMARKER RISK MODEL, filed on Nov. 25, 2014; U.S. patent application Ser. No. 15/998,427, SEPTIC SHOCK ENDOTYPING STRATEGY AND MORTALITY RISK FOR CLINICAL APPLICATION, filed on Aug. 15, 2018; U.S. Provisional Application No. 62/616,646, TEMPORAL ENDOTYPE TRANSITIONS REFLECT CHANGING RISK AND TREATMENT RESPONSE IN PEDIATRIC SEPTIC SHOCK, filed on Jan. 12, 2018; International Application No. PCT/US2017/032538, SIMPLIFICATION OF A SEPTIC SHOCK ENDOTYPING STRATEGY FOR CLINICAL APPLICATIONS, filed on May 12, 2017; U.S. Provisional Application No. 62/335,803, SIMPLIFICATION OF A SEPTIC SHOCK ENDOTYPING STRATEGY FOR CLINICAL APPLICATIONS, filed on May 13, 2016; U.S. Provisional Application No. 62/427,778, SIMPLIFICATION OF A SEPTIC SHOCK ENDOTYPING STRATEGY FOR CLINICAL APPLICATIONS, filed on Nov. 29, 2016; U.S. Provisional Application No. 62/428,451, SIMPLIFICATION OF A SEPTIC SHOCK ENDOTYPING STRATEGY FOR CLINICAL APPLICATIONS, filed on Nov. 30, 2016; U.S. Provisional Application No. 62/446,216, SIMPLIFICATION OF A SEPTIC SHOCK ENDOTYPING STRATEGY FOR CLINICAL APPLICATIONS, filed on Jan. 13, 2017; U.S. patent application Ser. No. 16/539,128, SEPTIC SHOCK ENDOTYPING STRATEGY AND MORTALITY RISK FOR CLINICAL APPLICATION, filed on Aug. 13, 2019; U.S. Provisional Application No. 62/764,831, Endotype Transitions During the Acute Phase of Pediatric Septic Shock Reflect Changing Risk and Treatment Response, filed on Aug. 15, 2018; U.S. Provisional Application No. 63/149,744, A CONTINUOUS METRIC TO ASSESS THE INTERACTION BETWEEN ENDOTYPE ASSIGNMENT AND CORTICOSTEROID RESPONSIVENESS IN SEPTIC SHOCK, filed on Feb. 16, 2021; International Patent Application No. PCT/US2022/016642, A CONTINUOUS METRIC TO ASSESS THE INTERACTION BETWEEN ENDOTYPE ASSIGNMENT AND CORTICOSTEROID RESPONSIVENESS IN SEPTIC SHOCK, filed on Feb. 16, 2022; U.S. Provisional Application No. 63/347,504, PREDICTING PERSISTENT MULTIPLE ORGAN DYSFUNCTION IN THE PEDIATRIC POPULATION AFTER CARDIOPULMONARY BYPASS USING SEPSIS PROGNOSTIC BIOMARKERS, filed on May 31, 2022; and U.S. Provisional Patent Application No. PEDIATRIC SEPSIS MULTIPLE ORGAN DYSFUNCTION SYNDROME RISK PREDICTION MODEL, filed on Jun. 1, 2022.

Unless otherwise noted, terms are to be understood according to conventional usage by those of ordinary skill in the relevant art.

As used herein, the term “sample” encompasses a sample obtained from a subject or patient. The sample can be of any biological tissue or fluid. Such samples include, but are not limited to, sputum, saliva, buccal sample, oral sample, blood, serum, mucus, plasma, urine, blood cells (e.g., white cells), circulating cells (e.g. stem cells or endothelial cells in the blood), tissue, core or fine needle biopsy samples, cell-containing body fluids, free floating nucleic acids, urine, stool, peritoneal fluid, and pleural fluid, tear fluid, or cells therefrom. Samples can also include sections of tissues such as frozen or fixed sections taken for histological purposes or micro-dissected cells or extracellular parts thereof. A sample to be analyzed can be tissue material from a tissue biopsy obtained by aspiration or punch, excision or by any other surgical method leading to biopsy or resected cellular material. Such a sample can comprise cells obtained from a subject or patient. In some embodiments, the sample is a body fluid that include, for example, blood fluids, serum, mucus, plasma, lymph, ascitic fluids, gynecological fluids, or urine but not limited to these fluids. In some embodiments, the sample can be a non-invasive sample, such as, for example, a saline swish, a buccal scrape, a buccal swab, and the like.

As used herein, “blood” can include, for example, plasma, serum, whole blood, blood lysates, and the like.

As used herein, the term “assessing” includes any form of measurement, and includes determining if an element is present or not. The terms “determining,” “measuring,” “evaluating,” “assessing” and “assaying” can be used interchangeably and can include quantitative and/or qualitative determinations.

As used herein, the term “monitoring” with reference to a patient following cardiopulmonary bypass (CPB) and at risk for persistent multiple organ dysfunction syndrome (MODS) refers to a method or process of determining various parameters of a patient's condition following CPB, including determining relevant biomarker expression levels at one or more points in time following CPB, to determine risk of persistent MODS and/or probability of mortality. In some embodiments, monitoring relates to a method or process of determining the therapeutic efficacy of a treatment being administered to a patient.

As used herein, “outcome” can refer to an outcome studied. In some embodiments in accordance with the present disclosure, “outcome” can refer to the presence of organ dysfunction, including persistent MODS, following CPB. In some embodiments, “outcome” can refer to two or more organ dysfunctions following CPB. In some embodiments, “outcome” can refer to cardiovascular, respiratory, renal, hepatic, hematologic, and neurologic dysfunction following CPB. In some embodiments, “outcome” referring to persistent MODS comprises cardiovascular, respiratory, renal, hepatic, hematologic, and/or neurologic dysfunction, and/or systemic inflammation. In some embodiments, “outcome” includes renal dysfunction, and/or increase in days requiring mechanical ventilatory support and cardiovascular support (e.g. use of vasoactive-inotropic infusion).

In some embodiments, “outcome” can include survival/mortality. The importance of survival/mortality following CPB is readily evident. In some embodiments, an increased risk for a poor outcome indicates that a therapy has had a poor efficacy, and a reduced risk for a poor outcome indicates that a therapy has had a good efficacy. Although mortality/survival is obviously an important outcome, survivors have clinically relevant short- and long-term morbidities that impact quality of life, which are not captured by the dichotomy of “alive” or “dead.”

As used herein, the terms “predicting outcome” and “outcome risk stratification” with reference to a patient following CPB refers to a method or process of prognosticating a patient's risk of a certain outcome. In some embodiments, predicting an outcome relates to monitoring the therapeutic efficacy of a treatment being administered to a patient. In some embodiments, predicting an outcome relates to determining a relative risk of an adverse outcome (e.g. complicated course) and/or mortality. In some embodiments, the predicted outcome is associated with administration of a particular treatment or treatment regimen. Such adverse outcome risk and/or mortality can be high risk, moderate risk, moderate-high risk, moderate-low risk, or low risk. Alternatively, such adverse outcome risk can be described simply as high risk or low risk, corresponding to high risk of adverse outcome (e.g. complicated course) and/or mortality probability, or high likelihood of therapeutic effectiveness, respectively. In some embodiments of the present disclosure, adverse outcome risk can be determined via the biomarker-based persistent MODS risk stratification as described herein. In some embodiments, predicting an outcome relates to determining a relative risk of persistent MODS following CPB. Such mortality risk can be high risk, moderate risk, moderate-high risk, moderate-low risk, or low risk. Alternatively, such mortality risk can be described simply as high risk or low risk, corresponding to high risk of death or high likelihood of survival, respectively. As related to the terminal nodes of the decision trees described herein, a “high risk terminal node” corresponds to an increased probability of adverse outcome (e.g. complicated course) and/or mortality according to a particular treatment or treatment regimen, whereas a “low risk terminal node” corresponds to a decreased probability of adverse outcome (e.g. complicated course) and/or mortality according to a particular treatment or treatment regimen.

As used herein, the term “high risk clinical trial” refers to one in which the test agent has “more than minimal risk” (as defined by the terminology used by institutional review boards, or IRBs). In some embodiments, a high risk clinical trial is a drug trial.

As used herein, the term “low risk clinical trial” refers to one in which the test agent has “minimal risk” (as defined by the terminology used by IRBs). In some embodiments, a low risk clinical trial is one that is not a drug trial. In some embodiments, a low risk clinical trial is one that that involves the use of a monitor or clinical practice process. In some embodiments, a low risk clinical trial is an observational clinical trial.

As used herein, the terms “modulated” or “modulation,” or “regulated” or “regulation” and “differentially regulated” can refer to both up regulation (i.e., activation or stimulation, e.g., by agonizing or potentiating) and down regulation (i.e., inhibition or suppression, e.g., by antagonizing, decreasing or inhibiting), unless otherwise specified or clear from the context of a specific usage.

As used herein, the term “subject” refers to any member of the animal kingdom. In some embodiments, a subject is a human patient. In some embodiments, a subject is a pediatric patient. In some embodiments, a pediatric patient is a patient under 18 years of age, while an adult patient is 18 or older. Unless stated otherwise, the terms “patient” or “child” (or “patients” or “children”) refer to a pediatric patient (i.e., under 18 years old).

As used herein, the terms “treatment,” “treating,” “treat,” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” can also encompass delivery of an agent or administration of a therapy in order to provide for a pharmacologic effect, even in the absence of a disease or condition.

As used herein, the term “marker” or “biomarker” refers to a biological molecule, such as, for example, a nucleic acid, peptide, protein, hormone, and the like, whose presence or concentration can be detected and correlated with a known condition, such as a disease state. It can also be used to refer to a differentially expressed gene whose expression pattern can be utilized as part of a predictive, prognostic or diagnostic process in healthy conditions or a disease state, or which, alternatively, can be used in methods for identifying a useful treatment or prevention therapy.

As used herein, the term “expression levels” refers, for example, to a determined level of biomarker expression. The term “pattern of expression levels” refers to a determined level of biomarker expression compared either to a reference (e.g. a housekeeping gene or inversely regulated genes, or other reference biomarker) or to a computed average expression value (e.g. in DNA-chip analyses). A pattern is not limited to the comparison of two biomarkers but is more related to multiple comparisons of biomarkers to reference biomarkers or samples. A certain “pattern of expression levels” can also result and be determined by comparison and measurement of several biomarkers as disclosed herein and display the relative abundance of these transcripts to each other.

As used herein, a “reference pattern of expression levels” refers to any pattern of expression levels that can be used for the comparison to another pattern of expression levels. In some embodiments of the disclosure, a reference pattern of expression levels is, for example, an average pattern of expression levels observed in a group of healthy or diseased individuals, serving as a reference group.

As used herein, the term “decision tree” refers to a standard machine learning technique for multivariate data analysis and classification. Decision trees can be used to derive easily interpretable and intuitive rules for decision support systems.

Sepsis and cardiopulmonary bypass (CPB) both cause cellular injury and release of molecules that activate the innate and adaptive immune responses resulting in pro-inflammatory mediator upregulation [1,3]. Research focusing on innate and adaptive immune gene expression and profiling in pediatric sepsis has generated the Pediatric Sepsis Biomarker Risk Model (PERSEVERE) [12-20], which is comprised of seven protein biomarkers of inflammation, and reliably predicts baseline risk of mortality and organ dysfunction among critically ill children with septic shock. PERSEVERE and, more recently, PERSEVERE II, have been utilized as risk-stratification tools to estimate probability of mortality and organ dysfunctions in pediatric septic patients [18].

Research on sepsis and CPB-mediated inflammation has identified significant overlap in inflammatory biomarker activation, including PERSEVERE biomarkers [5, 21-27]. The present study was therefore designed to study whether PERSEVERE biomarkers could be used to derive a unique risk model for early prediction of persistent multiple organ dysfunction syndrome (MODS) after CPB in pediatric patients.

As described herein, PERSEVERE biomarkers and clinical data were analyzed to determine if they can be combined to derive a new model to assess the risk of persistent CPB-related MODS in the early post-operative period. This study included 306 patients <18 years old admitted to a pediatric cardiac ICU after surgery requiring cardiopulmonary bypass (CPB) for congenital heart disease. Persistent MODS, defined as dysfunction of two or more organ systems on postoperative day 5, was the primary outcome. PERSEVERE biomarkers were collected 4 and 12 hours after CPB. Classification and Regression Tree methodology was used to derive a model to assess the risk of persistent MODS.

The successful model containing interleukin-8 (IL-8), chemokine ligand 3 (CCL3), and age as predictor variables, had an area under the receiver operating characteristic curve (AUROC) of 0.86 (0.81-0.91) for differentiating those with or without persistent MODS, and a negative predictive value of 99% (95-100). Ten-fold cross-validation of the model yielded a corrected AUROC of 0.75 (0.68-0.84).

Thus, a novel risk prediction model is provided to assess the risk for development of multiple organ dysfunction after pediatric cardiac surgery requiring CPB, using known clinical risk factors and biomarkers of inflammation originally identified as key markers of inflammation in pediatric patients with septic shock. IL-8 concentration was found to be the most predictive variable for development of MODS after CPB in the study patient population. Future studies can better define CPB related IL-8 pathophysiology and modifiable risk factors for IL-8 elevation after CPB.

This simple, biologically plausible model can accurately predict risk of persistent organ dysfunction in pediatric patients after cardiac surgery for congenital heart disease. In addition, this model can facilitate identification of a high-risk cohort to direct interventions and studies aimed at improving outcomes via mitigation of post-operative organ dysfunction.

### PERSEVERE-CPB and Applications Thereof

As described in the examples herein, inflammatory biomarkers and established clinical risk factors were used to derive a decision tree that is able to stratify patients by risk for developing persistent multiple organ dysfunction syndrome at post-operative day 5 after cardiopulmonary bypass surgery for congenital heart disease. Of the clinical risk factors and biomarkers included in this study, interleukin 8 (IL-8) concentration was found to be the most important predictor of persistent MODS.

PERSEVERE-CPB allows a heterogenous cardiac surgery population to be stratified into high, intermediate, and low risk groups based on risk for persistent MODS. The model functions exceptionally well in identifying low risk patients, as illustrated by a high negative predictive value and low negative likelihood ratio.

This model enables the clinician to increase vigilance in a smaller cohort of patients, which has added importance, as those falling into the high-risk PERSEVERE-CPB strata experienced worse clinical outcomes (longer duration of ventilator and vasoactive support, longer duration of stay, higher in-hospital morality) compared to the intermediate- and low-risk groups. This model can allow for early identification of patients categorized as low risk to receive standard of care supportive therapies, and those at intermediate or high risk to receive early targeted clinical interventions aimed at reducing the risk of MODS. Additionally, separation of low and higher risk cohorts can allow for prognostic enrichment in future clinical trials of interventions aimed at mitigating organ dysfunctions.

The development of a rapid point of care PERSEVERE biomarker panel allows for real time risk stratification, and there is ongoing work focused on the development of a rapid point of care PERSEVERE biomarker panel, which will expand the utility of PERSEVERE-CPB, Once available, PERSEVERE-CPB can be implemented in efforts to improve postoperative outcomes, including reduction of MODS. This also allows real-time physiologic and laboratory data to be incorporated into the model to improve the precision and specificity.

For assessing risk of persistent MODS, PERSEVERE-CPB performed well when compared to existing pediatric critical care and cardiac surgery risk-assessment tools (STAT, PRISM III, PELOD 2). In particular, PERSEVERE-CPB performed similarly to the postoperative day one PELOD-2 score for predicting development of persistent MODS. Although STAT and PRISM III were primarily validated to predict risk of mortality and not MODS, the low mortality rate in our cohort did not allow us to develop a biomarker-based predictive model for in-hospital mortality.

IL-8 level functioned as the upper level decision rule, indicating that it plays a key role in determination of risk for MODS. Almost 42% of patients who developed persistent MODS fell into terminal node 4, with an elevated 12 hour IL-8 concentration. IL-8 is one of the more studied biomarkers of inflammation in patients after CPB. It is a neutrophil chemoattractant, plays a pivotal role in neutrophil activation, and is produced in large quantities by endothelial cells [40]. Elevated postoperative IL-8 has been associated with markers of low cardiac output (low mixed venous oxygen concentration and higher inotropic score) [41], development of postoperative acute kidney injury [26,42,43], increased duration of mechanical ventilation [22,43], and longer ICU length of stay [6]. The pathophysiologic role IL-8 plays in neutrophil/endothelium activation, bypass-mediated inflammation, and development of MODS warrants further examination, with obvious potential as a therapeutic target.

In comparison, CCL3, or macrophage inflammatory protein 1α (MIP-1α), has not been extensively studied in bypass-mediated inflammation. During acute inflammation, CCL3 aids in the recruitment of leukocytes and plays a role in neutrophil infiltration [45,46]. Since both PERSEVERE and PERSEVERE-II have demonstrated CCL3 plays a major role in discrimination of both mortality and multiple organ failure in severe pediatric sepsis [47], further investigation into the role of CCL3 in CPB-mediated inflammation and its contribution to development of organ dysfunction is warranted.

Age less than 12 months at time of surgery functioned as the second level decision rule in PERSEVERE-CPB. Younger age is known to be associated with increased morbidity after pediatric cardiac surgery [36,37,48], which is not a surprise given that infants and neonates undergo the most complex and highest risk surgeries. Future efforts to create risk models specific to infants and neonates can help determine if there are modifiable risk factors or potential therapeutic targets or if their increased risk is attributable to complexity of surgery and cardiac physiology (such as single ventricle physiology) alone.

Perioperative steroids are used in children undergoing CPB to blunt the bypass-mediated inflammatory response [49]. Interestingly, the majority of the high risk cohort (17 out of 20 subjects) were hospitalized neonates and infants, which indicates that inflammation can have a bigger impact in outcome in this subset of patients, despite receiving two doses of steroids. The high risk cohort was more likely to receive steroids for hypotension in the first 24 hours postoperative, which can reflect an enhanced inflammatory response leading to higher degree or longer lasting vasoplegia (Table 6).

Unlike prior studies, use of dialysis was associated with increased IL-8 at both 4 and 12 hours post-CPB in both the entire cohort and the neonatal subpopulation. CCL-3 concentrations were higher in the dialysis group, but only 12 hour concentrations in the entire cohort were significant, as shown in Table 7. Future studies can be designed to evaluate postoperative inflammatory biomarker concentrations over time, use of dialysis, and correlation with risk of persistent MODS, particularly in the neonates and infants who, in this study, comprise a majority of the most at risk population.

Cross-validation AUC for the PERSEVERE-CPB model showed good ability to predict persistent MODS, comparable to postoperative PRISM III and PELOD-2.

### Additional Patient Information

The demographic data, clinical characteristics, and/or results from other tests or indicia of MODS specific to a pediatric patient following CPB can affect the patient's outcome risk. Accordingly, such demographic data, clinical characteristics, and/or results from other tests or indicia of MODS can be incorporated into the methods described herein which allow for stratification of individual pediatric patients in order to determine the patient's outcome risk. Such demographic data, clinical characteristics, and/or results from other tests or indicia of MODS can also be used in combination with the methods described herein which allow for stratification of individual pediatric patients in order to determine the patient's outcome risk.

Such pediatric patient demographic data can include, for example, the patient's age, race, ethnicity, gender, and the like. In some embodiments, the biomarker-based persistent MODS following CPB risk stratification described herein can incorporate or be used in combination with the patient's age, race, ethnicity, and/or gender to determine an outcome risk.

Such patient clinical characteristics and/or results from other tests or indicia of MODS can include, for example, the patient's co-morbidities, and the like.

Patient co-morbidities can include, for example, acute lymphocytic leukemia, acute myeloid leukemia, aplastic anemia, atrial and ventricular septal defects, bone marrow transplantation, caustic ingestion, chronic granulomatous disease, chronic hepatic failure, chronic lung disease, chronic lymphopenia, chronic obstructive pulmonary disease (COPD), congestive heart failure (NYHA Class IV CHF), Cri du Chat syndrome, cyclic neutropenia, developmental delay, diabetes, DiGeorge syndrome, Down syndrome, drowning, end stage renal disease, glycogen storage disease type 1, hematologic or metastatic solid organ malignancy, hemophagocytic lymphohistiocytosis, hepatoblastoma, heterotaxy, hydrocephalus, hypoplastic left heart syndrome, IPEX Syndrome, kidney transplant, Langerhans cell histiocytosis, liver and bowel transplant, liver failure, liver transplant, medulloblastoma, metaleukodystrophy, mitochondrial disorder, multiple congenital anomalies, multi-visceral transplant, nephrotic syndrome, neuroblastoma, neuromuscular disorder, obstructed pulmonary veins, Pallister Killian syndrome, Prader-Willi syndrome, requirement for chronic dialysis, requirement for chronic steroids, retinoblastoma, rhabdomyosarcoma, rhabdosarcoma, sarcoma, seizure disorder, severe combined immune deficiency, short gut syndrome, sickle cell disease, sleep apnea, small bowel transplant, subglottic stenosis, tracheal stenosis, traumatic brain injury, trisomy 18, type 1 diabetes mellitus, unspecified brain tumor, unspecified congenital heart disease, unspecified leukemia, VATER Syndrome, Wilms tumor, and the like. Any one or more of the above patient co-morbidities can be indicative of the presence or absence of chronic disease in the patient.

In some embodiments, the biomarker-based persistent MODS following CPB risk stratification as described herein can incorporate the patient's co-morbidities to determine an outcome risk and/or mortality probability. In some embodiments, the biomarker-based persistent MODS following CPB risk stratification as described herein can be used in combination with the patient's co-morbidities to determine an outcome risk and/or mortality probability.

### PERSEVERE, PERSEVERE II, and Other Population-Based Risk Scores

As mentioned previously, the PERSEVERE model for estimating baseline mortality risk in children with septic shock was previously derived and validated. PERSEVERE is based on a panel of 12 serum protein biomarkers measured from blood samples obtained during the first 24 hours of a septic shock diagnosis, selected from among 80 genes having an association with mortality risk in pediatric septic shock. Of those 12 serum biomarkers, the derived and validated PERSEVERE model is based on Interleukin-8 (IL-8), Heat shock protein 70 kDA (HSP70), C—C Chemokine ligand 3 (CCL3), C—C Chemokine ligand 4 (CCL4), Granzyme B (GZMB), Interleukin-1α (IL-1α), and Matrix metallopeptidase 8 (MMP8). PERSEVERE additionally takes patient age into account.

The PERSEVERE decision tree has 8 terminal nodes. Of these, 3 terminal nodes of the PERSEVERE decision tree are determined to be low risk/low mortality probability (terminal nodes 2, 4, and 7), while 5 terminal nodes of the PERSEVERE decision tree are determined to be intermediate to high risk/high mortality probability (terminal nodes 1, 3, 5, 6, and 8). In some embodiments, a low risk/low mortality probability terminal nodes has a mortality probability between 0.000 and 0.025, while an intermediate to high risk/high mortality probability terminal nodes has a mortality probability greater than 0.025.

In some embodiments of the present disclosure, a patient sample is analyzed for the PERSEVERE serum protein biomarkers IL-8 and HSP70, as well as for the endothelial biomarkers ICAM-1, Thrombomodulin, Angpt-2/Angpt-1, and/or Angpt-2/Tie-2.

In some embodiments of the present disclosure, the PERSEVERE mortality probability stratification can be used in combination with the biomarker-based persistent MODS following CPB risk stratification as described herein. In some embodiments, the biomarker-based persistent MODS following CPB risk stratification, as described herein, can be used in combination with a patient endotyping strategy and/or Z score determination. In some embodiments, the combination of a biomarker-based persistent MODS following CPB risk stratification, with an endotyping strategy and/or Z score determination, can be used to determine an appropriate treatment regimen for a patient. For example, such combinations can be used to identify which patients are more likely to benefit from one or more high risk therapies or rather from standard of care treatment.

As mentioned previously, the PERSEVERE II model for estimating baseline mortality risk in children with septic shock was previously derived and validated. PERSEVERE II is based on a panel of 5 serum protein biomarkers measured from blood samples obtained during the first 24 hours of a septic shock diagnosis. Of those 5 serum biomarkers, the derived and validated PERSEVERE II model is based on interleukin-8 (IL-8), C—C chemokine ligand 3 (CCL3), and heat shock protein 70 kDa 1B (HSPA1B), as well as platelet count.

The PERSEVERE II decision tree has 5 terminal nodes. Of these, 3 terminal nodes of the PERSEVERE II decision tree are determined to be low risk/low mortality probability (terminal nodes 1, 2, and 4), while 2 terminal nodes of the PERSEVERE II decision tree are determined to be intermediate to high risk/high mortality probability (terminal nodes 3 and 5). In some embodiments, a low risk/low mortality probability terminal nodes has a mortality probability between 0.000 and 0.025, while an intermediate to high risk/high mortality probability terminal nodes has a mortality probability greater than 0.025.

In some embodiments of the present disclosure, a patient sample is analyzed for the PERSEVERE II serum protein biomarkers IL-8, CCL3, and HSPA1B, and platelet count, as well as for the endothelial biomarkers Tie-2, Angpt-2, and sTM.

In some embodiments of the present disclosure, the PERSEVERE II mortality probability stratification can be used in combination with the biomarker-based persistent MODS following CPB risk stratification as described herein. In some embodiments, the biomarker-based persistent MODS following CPB risk stratification, as described herein, can be used in combination with a patient endotyping strategy and/or Z score determination. In some embodiments, the combination of a biomarker-based persistent MODS following CPB risk stratification, with an endotyping strategy and/or Z score determination, can be used to determine an appropriate treatment regimen for a patient. For example, such combinations can be used to identify which patients are more likely to benefit from one or more high risk therapies, or rather from standard of care treatment.

A number of additional models that generate mortality prediction scores based on physiological variables have been developed to date. These can include the PRISM, Pediatric Index of Mortality (PIM), and/pediatric logistic organ dysfunction (PELOD) models, and the like.

Such models can be very effective for estimating population-based outcome risks but are not intended for stratification of individual patients. The methods described herein which allow for stratification of individual patients can be used alone or in combination with one or more existing population-based risk scores.

In some embodiments, the biomarker-based persistent MODS following CPB risk stratification described herein can be used with one or more additional population-based risk scores. In some embodiments, the biomarker-based persistent MODS following CPB risk stratification described herein can be used in combination with PRISM. In some embodiments, the biomarker-based persistent MODS following CPB risk stratification described herein can be used in combination with PIM. In some embodiments, the biomarker-based persistent MODS following CPB risk stratification herein can be used in combination with PELOD. In some embodiments, the biomarker-based persistent MODS following CPB risk stratification described herein can be used in combination with a population-based risk score other than PRISM, PIM, and PELOD.

### High Risk Therapies

High risk, invasive therapeutic and support modalities can be used to treat patients at risk of developing persistent MODS. The methods described herein which allow for the patient's outcome risk to be determined can help inform clinical decisions regarding the application of high risk therapies to specific pediatric patients, based on the patient's outcome risk.

High risk therapies include, for example, adjuvant hemoperfusion, plasma filtration and adsorption therapies, extracorporeal membrane oxygenation/life support, plasmapheresis, pulmonary artery catheterization, high volume continuous hemofiltration, and the like. High risk therapies can also include non-corticosteroid therapies, e.g. alternative therapies and/or other high risk therapies. In particular, patients at high risk of persistent MODS following CPB can be treated with immune enhancing therapies, such as, for example, recombinant human thrombomodulin, Angiopoietin-2 inhibitors, Tie-2 agonists, and the like. Patients at high risk of persistent MODS following CPB can also be treated with specific IL-8 targeting therapy once such treatments are developed and available.

High risk therapies can also include steroids, such as corticosteroids (e.g. methylprednisolone, hydrocortisone), for treating hypotension and/or reducing inflammation secondary to CPB. Additional measures to reduce CPB-mediated inflammation can also include modified ultrafiltration after surgery while still on bypass and peritoneal dialysis.

High risk therapies can also include peritoneal dialysis, which has been shown to decrease inflammatory biomarkers in the neonatal population after CPB.

In some embodiments, individualized treatment can be provided to a pediatric patient by selecting a pediatric patient classified as high risk by the methods described herein for one or more high risk therapies. In some embodiments, individualized treatment can be provided to a pediatric patient by excluding a pediatric patient classified as low risk from one or more high risk therapies.

Certain embodiments of the disclosure include using quantification data from a gene-expression analysis and/or from a protein, mRNA, and/or DNA analysis, from a sample of blood, urine, saliva, broncho-alveolar lavage fluid, or the like. Embodiments of the disclosure include not only methods of conducting and interpreting such tests but also include reagents, compositions, kits, tests, arrays, apparatuses, processing devices, assays, and the like, for conducting the tests. The compositions and kits of the present disclosure can include one or more components which enable detection of the biomarkers disclosed herein and combinations thereof and can include, but are not limited to, primers, probes, cDNA, enzymes, covalently attached reporter molecules, and the like.

Diagnostic-testing procedure performance is commonly described by evaluating control groups to obtain four critical test characteristics, namely positive predictive value (PPV), negative predictive value (NPV), sensitivity, and specificity, which provide information regarding the effectiveness of the test. The PPV of a particular diagnostic test represents the proportion of positive tests in subjects with the condition of interest (i.e. proportion of true positives); for tests with a high PPV, a positive test indicates the presence of the condition in question. The NPV of a particular diagnostic test represents the proportion of negative tests in subjects without the condition of interest (i.e. proportion of true negatives); for tests with a high NPV, a negative test indicates the absence of the condition. Sensitivity represents the proportion of subjects with the condition of interest who will have a positive test; for tests with high sensitivity, a positive test indicates the presence of the condition in question. Specificity represents the proportion of subjects without the condition of interest who will have a negative test; for tests with high specificity, a negative test indicates the absence of the condition.

The threshold for the disease state can alternatively be defined as a 1-D quantitative score, or diagnostic cutoff, based upon receiver operating characteristic (ROC) analysis. The quantitative score based upon ROC analysis can be used to determine the specificity and/or the sensitivity of a given diagnosis based upon subjecting a patient to a decision tree described herein in order to predict an outcome for a pediatric patient with following CPB.

The correlations disclosed herein, between pediatric patient septic shock biomarker levels and/or mRNA levels and/or gene expression levels, and/or protein expression levels, combined with the patient age, provide a basis for conducting a stratification of patients following CPB and at risk of developing persistent MODS, or for enhancing the reliability of a diagnosis of persistent MODS, by combining the results of a quantification of a septic shock biomarker with results from other tests or indicia of persistent MODS, or for determining an appropriate treatment regimen for a pediatric patient following CPB and at risk for developing persistent MODS. For example, the results of a quantification of one biomarker could be combined with the results of a quantification of one or more additional biomarker, protein, cytokine, mRNA, or the like. Thus, even in situations in which a given biomarker correlates only moderately or weakly with risk of persistent MODS, providing only a relatively small PPV, NPV, specificity, and/or sensitivity, the correlation can be one indicium, combinable with one or more others that, in combination, provide an enhanced clarity and certainty of diagnosis. Accordingly, the methods and materials of the disclosure are expressly contemplated to be used both alone and in combination with other tests and indicia, whether quantitative or qualitative in nature.

Having described the various embodiments in detail, it will be apparent that modifications, variations, and equivalent embodiments are possible without departing from the scope of the embodiments defined in the appended claims. Furthermore, it should be appreciated that all examples in the present disclosure are provided as non-limiting examples.

### EXAMPLES

The following non-limiting examples are provided to further illustrate embodiments disclosed herein. It should be appreciated by those of skill in the art that the techniques disclosed in the examples that follow represent approaches that have been found to function well in the practice of the embodiments disclosed herein, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of those embodiments.

### Example 1

### Methods

### Patients, Samples and Data Collection:

The study was approved by the Institutional Review Board at Cincinnati Children's Hospital Medical Center.

All patients under the age of 18 years old undergoing surgery requiring CPB for correction of congenital heart disease between November 2016 and November 2020 were screened for eligibility. Patients were only included for their index surgery to prevent re-enrollment of patients requiring reoperation for residual lesions while still recovering from their initial surgery. For patients with single ventricle physiology, each surgical stage was treated as a separate index surgery, i.e., stage 1 palliative surgery, Glenn operation, Fontan operation, and/or biventricular repair. Due to the short time frame between stage 1 and Glenn, Glenn candidates were screened prior to re-enrollment and were excluded if they met criteria for organ dysfunction at time of screening. Patients undergoing CPB for heart or lung transplantation, patients requiring immunosuppression, and patients with suspected or proven infection were excluded.

Three-hundred and fifty-nine patient encounters (293 unique patients) were consented for the study. Of these, 306 encounters were included in the analysis, because both 4 and 12 hour biomarker samples were collected within the specified time. Baseline demographic, clinical, and laboratory data used to calculate severity of illness scoring and determine organ dysfunction were extracted from the electronic medical record (EMR). To minimize clinically unnecessary blood draws, laboratory data to assess for organ dysfunction was only collected at discretion of the managing clinical team.

### Patient and Disease Evaluation:

The Society of Thoracic Surgery-European Association for Cardiothoracic Surgery (STAT) mortality category [28,29] was used to account for risk related to surgical complexity. Pre- and postoperative severity of illness was assessed using Pediatric Risk of Mortality score III (PRISM III) [30]. Organ dysfunction was defined via adaption of Goldstein criteria to account for differences in the postoperative congenital heart disease population when compared to the pediatric sepsis population, as shown in Table 1. Persistent MODS was defined a priori as dysfunction of 2 or more organ systems on postoperative day 5. As an additional measure of organ dysfunction, daily Pediatric Logistic Organ Dysfunction-2 [PELOD-2] scores were calculated preoperatively and for the first 5 postoperative days [32,33].

TABLE 1
Definitions of organ dysfunction.

| Organ System | Definition of Dysfunction |
|---|---|
| Cardiovascular | On vasoactive drugs by POD 5 |
| | or persistent lactatemia >5 mmol/L |
| | or hypotension <5^{th} percentile for age or systolic blood pressure <2 SD below normal |
| | for age |
| Respiratory | Use of invasive or non-invasive ventilation by POD 5 |
| | or persistent respiratory acidosis with PaCO2 >65 mmHg or 20 mmHg above baseline |
| | or PaO2/FiO2 <300 torr in absence of cyanotic heart disease or preexisting lung disease |
| Renal | Cr >2 times upper limit of normal for age or 2-fold increase in baseline Cr, use of |
| | dialysis |
| GI/Hepatic | Total bilirubin >4 mg/dL (outside of newborn period) or ALT 2 times upper normal limit |
| | for age, development of NEC |
| Hematologic | Platelet count <80,000/mm³ |
| | or INR >2 in a patient not on warfarin |
| Neurologic | GCS <11 in a non-sedated patient |
| | or acute mental status change with decrease in GCS of >3 from baseline |
| | or new cerebrovascular accident |

POD: post-operative day;
SD: standard deviation;
PaCO2: arterial partial pressure of carbon dioxide;
PaO2: arterial partial pressure of oxygen;
FiO2: fraction of inspired oxygen;
Cr: creatinine;
ALT: alanine transaminase;
NEC: necrotizing enterocolitis;
INR: international normalized ratio;
GCS: Glascow coma score

### Clinical and Surgical Management:

All patients received methylprednisolone (30 mg/kg) as part of the CPB circuit prime. Neonates and patients in the hospital prior to their scheduled operation received an additional dose of methylprednisolone (30 mg/kg) the morning of surgery (prior to CPB initiation). Choice of anesthesia was not standardized and left to the decision of the cardiac anesthesiologist. All patients received either modified ultrafiltration and/or continuous ultrafiltration intraoperatively, based on surgeon preference. The use of additional steroids and use of postoperative peritoneal dialysis was left to the discretion of the clinical team.

### Biomarker Collection:

Biomarkers were collected 4 and 12 hours post-CPB, based on studies suggesting peak inflammation occurs within 24 hours of CPB separation [4-6, 8, 21, 34]. Blood was collected within a +/−60 minute window, spun down to serum, and stored at −80 C until ready to be analyzed. Seven PERSEVERE biomarkers were measured in this study: granzyme B (GZMB), heat shock protein 70 kDa 1B (HSP70, also referred to as HSPA1B), interleukin 1α (IL-1α), interleukin 8 (IL-8), C—C chemokine ligand 3 (CCL3), C—C chemokine ligand 4 (CCL4), matrix metalloproteinase 8 (MMP-8). Serum biomarker concentrations were measured according to manufacturer's instructions using the HSP2MAG-63K multiplex bead platform (MILLIPLEX MAP Human Sepsis Magnetic Bead Panel 2-Immune Response Multiplex Assay) designed by the EMD Millipore Corporation (Billerica, MA, USA).

### Statistical Analysis:

Descriptive statistical analyses were performed using R (version 4.0.4). Demographic, clinical, and biomarker data were described using medians with interquartile ranges (IQR), means with standard deviations, or frequencies with percentages as appropriate. Comparisons of data for patients with and without persistent MODS were performed using the Kruskal-Wallis, chi-squared, or Fisher's exact tests as appropriate. Multivariate regression analysis, controlling for clinical data, was performed to examine the relationship between biomarker concentrations at 4 and 12 hours and risk for development of MODS.

Classification and regression tree (CART) analysis was used to determine biomarker cut-points and derive a decision tree (Salford Predictive Modeler v6.6, Salford Systems, San Diego, CA) [35]. Candidate prediction variables for derivation of the decision tree were as follows: all seven PERSEVERE biomarkers at 4 and 12 hour time points, change in PERSEVERE biomarker levels from 4 to 12 hours, age in months (included as both a continuous and dichotomous variable), single ventricle status, history of prematurity, CPB time, maximum vasoactive inotropic score (VIS) and STS-EACTS mortality category. Clinical predictor variable selection was based on extant literature [36-39]. Tuning parameters determined a priori included: 10-fold cross validation, at least one of the paired terminal daughter nodes contains ≥5% of the subjects in the root node, and no predictor variables repeated within one of the two main branches.

Performance of the decision tree was determined by generating a classification table of true versus predicted status and calculation of discrimination metrics including sensitivity, specificity, positive and negative predictive values, and area under the receiver operating curve (AUROC). The prediction model, referred to herein as PERSEVERE-CPB, was compared to PRISM III and STS-EACTS mortality category, as they are widely accepted and validated risk assessment and severity of illness scoring systems this patient population, using the AUROC, sensitivity, and specificity. PERSEVERE-CPB was further compared to the 24-hour postoperative PELOD-2 score, as PELOD-2 is a validated scoring system for organ dysfunction [32].

Using risk categories (referred to as PERSEVERE-CPB risk category), stratified the cohort into risk category based on high, intermediate, and low risk terminal nodes of the model. The association of risk category with administration of postoperative steroids was then evaluated for hypotension and clinical outcomes.

Finally, an uncontrolled subanalysis was performed comparing biomarker concentrations in subjects who received dialysis (peritoneal or continuous renal replacement therapy) within the first 24 hours after surgery to assess the potential effect of dialysis on biomarker concentration.

### Example 2

### Patient Cohorts

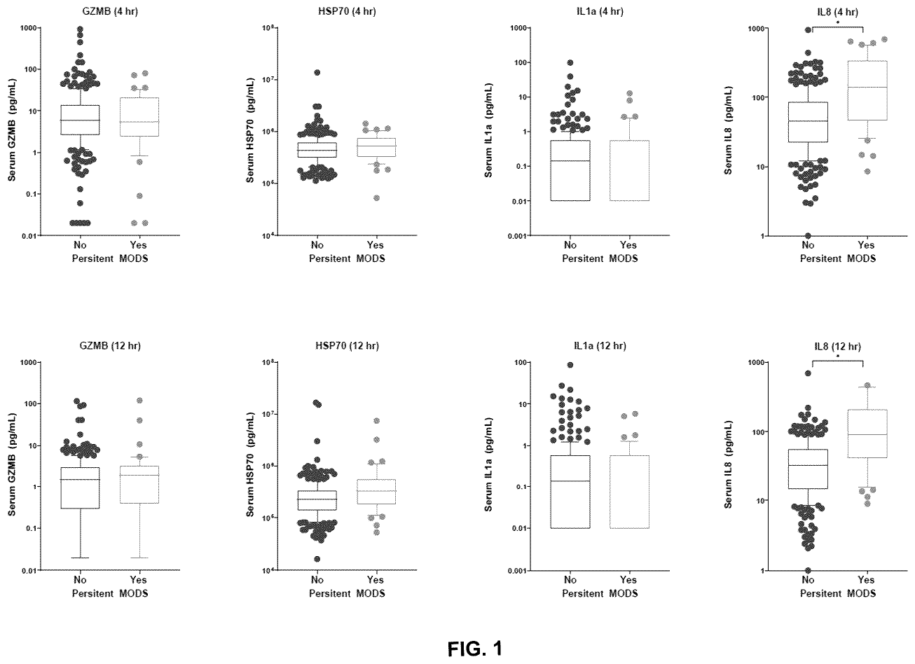

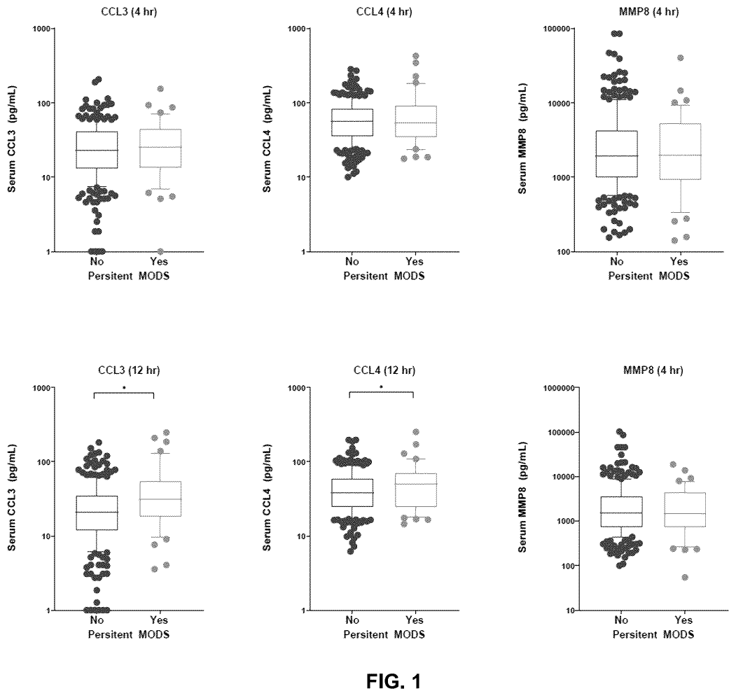

Demographics, clinical characteristic, and biomarker concentrations of patients with and without persistent MODS are shown in Table 2, Table 3, Table 4, and FIG. 1. As shown in FIG. 1, the serum interleukin-8 (IL-8) concentration was significantly elevated at 4 hours after separation from cardiopulmonary bypass (CPB) in patients who developed persistent MODS and those who did not. IL-8, CCL-3 and CCL-4 concentrations at 12 hours after separation from CPB were also significantly elevated in the cohort that developed persistent MODS compared to those that did not. Biomarker abbreviations displayed are as follows: GZMB, granzyme B; HSPA1B, heat shock protein 70 kDa 1B; IL-1α, interleukin 1α; IL-8, interleukin 8; CCL3, C—C chemokine ligand 3; CCL4, C—C chemokine ligand 4; MMP-8, matrix metalloproteinase 8.

Of the 306 subjects with biomarkers drawn at both 4 and 12 hours after separation from CPB, 43 (14.1%) had persistent MODS on POD 5. The cohort with persistent MODS was significantly younger, had a history of prematurity, had higher illness severity before and immediately after CPB, received more organ support, were more likely to receive steroids for post-operative hypotension, and had worse clinical outcomes. In multivariate logistic regression models, accounting for age less than 12 months, STAT mortality category, CPB time, and single ventricle status, IL-8 concentration at both 4 and 12 hours were independently associated with risk of persistent MODS, as did 12-hour concentrations of GZMB and CCL3, as shown in Table 3.

TABLE 2
Demographics and clinical characteristics.

| | All* | | MODS* | | No MODS* | | p value |
|---|---|---|---|---|---|---|---|
| Number of subjects (%) | 306 | | 43 | (14.0) | 263 | (86.0) | — |
| Age (months) | 6 | (3-42.9) | 2 | (0.2-5.3) | 8 | (3.9-48) | <0.001 |
| Number of females (%) | 134 | (43.8) | 20 | (46.5) | 114 | (43.3) | 0.7 |
| Race, number (%) | | | | | | | |
| White, non-Hispanic | 269 | (87.9) | 34 | (79.1) | 235 | (89.3) | 0.31 |
| White, Hispanic | 6 | (2.0) | 2 | (4.7) | 4 | (1.5) | |
| Black | 23 | (7.5) | 6 | (13.9) | 17 | (6.5) | |
| Other | 8 | (2.6) | 1 | (2.3) | 7 | (2.7) | |
| Number of neonates | 43 | (14.1) | 17 | (39.5) | 26 | (9.9) | <0.001 |
| (%) | | | | | | | |
| Number of single | 117 | (38.2) | 23 | (53.5) | 94 | (35.7) | 0.026 |
| ventricle patients (%) | | | | | | | |
| Number of infants (%) | 182 | (59.5) | 38 | (88.4) | 144 | (54.8) | <0.001 |
| Number of infants born | 45 | (14.7) | 13 | (30.2) | 32 | (12.2) | 0.002 |
| premature (%) | | | | | | | |
| STAT, number (%) | | | | | | | |
| 1 | 47 | (15.4%) | 3 | (7.0) | 44 | (16.7) | <0.001 |
| 2 | 131 | (42.8%) | 12 | (27.9) | 119 | (45.2) | |
| 3 | 46 | (15.2%) | 3 | (7.0) | 43 | (16.3) | |
| 4 | 60 | (19.6%) | 15 | (34.9) | 45 | (17.1) | |
| 5 | 22 | (7.2%) | 10 | (23.2) | 12 | (4.6) | |
| CPB time in minutes | 138.0 | (92.3; 183.0) | 176.0 | (112.0; 206.5) | 132.0 | (89.0; 179.0) | 0.005 |
| Number receiving MUF | 195 | (63.7) | 30 | (69.8) | 165 | (62.7) | 0.374 |
| (%) | | | | | | | |
| Pre-op PRISM III | 2.0 | (0.0; 3.0) | 5.0 | (3.0; 7.0) | 0.0 | (0.0; 3.0) | <0.001 |
| Post-op PRISM III | 8.0 | (6.0; 12.0) | 13.0 | (10.0; 16.0) | 8.0 | (5.0; 11.0) | <0.001 |
| PELOD-2 preoperative | 0.0 | (0.0; 2.0) | 2.0 | (0.0; 2.0) | 0.0 | (0.0; 2.0) | <0.001 |
| PELOD-2 24 hours | 4.0 | (2.0; 6.0) | 7.0 | (5.0; 8.0) | 4.0 | (2.0; 5.0) | <0.001 |
| postoperative | | | | | | | |
| VIS at 4 hours post- | 7.0 | (5.0; 10.0) | 8.0 | (7.0; 11.8) | 7.0 | (4.5; 9.0) | 0.007 |
| CPB | | | | | | | |
| Maximum VIS | 7.0 | (5.0; 15.4) | 17.5 | (14.5; 26.0) | 7.0 | (5.0; 12.5) | <0.001 |
| Lowest pH | 7.29 | (7.26-7.33) | 7.25 | (7.2; 7.3) | 7.3 | (7.3; 7.3) | <0.001 |
| Peak lactate | 2.4 | (1.6-4.0) | 3.7 | (2.3; 6.3) | 2.2 | (1.5; 3.8) | <0.001 |
| Number receiving PD | 12 | (3.9) | 9 | (20.9) | 3 | (1.1) | <0.001 |
| or CRRT postoperative | | | | | | | |
| (%) | | | | | | | |
| Number receiving | 70 | (22.9) | 24 | (55.8) | 46 | (17.5) | <0.001 |
| steroids postoperative | | | | | | | |
| (%) | | | | | | | |
| Number receiving | 27 | (8.8) | 13 | (30.2) | 14 | (5.3) | <0.001 |
| steroids for hypotension | | | | | | | |
| postoperative (%) | | | | | | | |
| Ventilator-free days | 27.0 | (26.0; 28.0) | 17.0 | (13.0; 23.0) | 28.0 | (26.0; 28.0) | <0.001 |
| Vasoactive-free days | 26.0 | (25.0; 27.0) | 20.0 | (14.5; 22.0) | 27.0 | (26.0; 27.0) | <0.001 |
| Number of in-hospital | 7 | (2.3) | 6 | (14.0) | 1 | (0.4) | <0.001 |
| mortality (%) | | | | | | | |
| Number alive and out | 267 | (87.3) | 20 | (46.5) | 247 | (93.9) | <0.001 |
| of the hospital | | | | | | | |
| by POD 28 (%) | | | | | | | |
| CICU LOS | 3.0 | (2.0; 8.0) | 15.0 | (11.0; 34.0) | 3.0 | (2.0; 4.5) | <0.001 |
| Hospital LOS | 7.0 | (4.0; 15.0) | 24.0 | (19.0; 67.0) | 7.0 | (4.0; 11.0) | <0.001 |

All data is presented as median (interquartile range) unless specified;
MODS: persistent multiple organ dysfunction at postoperative day 5;
neonate: <30 days old;
infant: <12 month old;
STAT: Society of Thoracic Surgery-European Association for Cardiothoracic Surgery mortality category;
CPB: cardiopulmonary bypass;
PRISM: Pediatric Risk of Mortality score;
PELOD-2: Pediatric Logistic Organ Dysfunction Score-2;
VIS: vasoactive inotropic score;
POD: postoperative day;
CICU: cardiac intensive care unit;
LOS: length of stay

TABLE 3
Development of MODS based on PERSEVERE biomarkers.

| | |
|---|---|
| OR (95% CI) | p-value |

Biomarkers at 4 hours

| | | |
|---|---|---|
| GZMB | 0.79 (0.20; 1.18) | 0.524 |
| HSP70 | 0.94 (0.35; 1.35) | 0.852 |
| IL-1α | 1.08 (0.50; 1.45) | 0.684 |
| IL-8 | 1.94 (1.41; 2.77) | <0.001 |
| CCL3 | 1.07 (0.74; 1.49) | 0.7 |
| CCL4 | 1.21 (0.88; 1.64) | 0.21 |
| MMP-8 | 1.15 (0.69; 1.60) | 0.473 |

Biomarkers at 12 hours

| | | |
|---|---|---|
| GZMB | 1.42 (1.04; 1.88) | 0.012 |
| HSP70 | 1.27 (0.89; 1.65) | 0.081 |
| IL-1α | 0.69 (0.18; 1.23) | 0.394 |
| IL-8 | 11.42 (2.91; 57.11) | 0.001 |
| CCL3 | 1.36 (1.02; 1.84) | 0.038 |
| CCL4 | 1.27 (0.92; 1.71) | 0.125 |
| MMP-8 | 0.99 (0.46; 1.44) | 0.963 |

Odds ratios (OR) obtained via logistic regression.
Each biomarker was modeled separately.
All models adjusted for age less than 12 months (infant), STAT mortality category, single ventricle status, and time (in minutes) on cardiopulmonary bypass.
CI: confidence interval; GZMB, granzyme B; HSPA1B, heat shock protein 70 kDa 1B; IL-1α, interleukin 1α; IL-8, interleukin 8; CCL3, C-C chemokine ligand 3; CCL4, C-C chemokine ligand 4; MMP-8, matrix metalloproteinase 8.

TABLE 4
Univariate association between PERSEVERE biomarkers
and risk of Persistent MODS among children
undergoing cardiopulmonary bypass.

| | |
|---|---|
| OR (95% CI) | p-value |

Biomarkers at 4 hours

| | | |
|---|---|---|
| GZMB | 0.81 (0.27; 1.20) | 0.55 |
| HSP70 | 1.01 (0.61; 1.31) | 0.93 |
| IL-1α | 0.95 (0.44; 1.26) | 0.81 |
| IL-8 | 2.42 (1.78; 3.42) | <0.001 |
| CCL3 | 1.13 (0.82; 1.49) | 0.42 |
| CCL4 | 1.28 (0.96; 1.68) | 0.07 |
| MMP-8 | 0.91 (0.55; 1.25) | 0.66 |

Biomarkers at 12 hours

| | | |
|---|---|---|
| GZMB | 1.14 (0.85; 1.45) | 0.3 |
| HSP70 | 1.12 (0.82; 1.42) | 0.37 |
| IL-1α | 0.68 (0.16; 1.18) | 0.46 |
| IL-8 | 32.97 (8.73; 154.67) | <0.001 |
| CCL3 | 1.60 (1.23; 2.11) | 0.001 |
| CCL4 | 1.31 (0.98; 1.73) | 0.05 |
| MMP-8 | 0.83 (0.41; 1.20) | 0.48 |

Odd ratio (OR) with 95% confidence intervals (95% CI) obtained via logistic regression.
Each biomarker was modeled separately.
ORs scaled to reflect one standard deviation increase in concentration (pg/mL).
MODS: persistent multiple organ dysfunction at postoperative day 5;
GZMB, granzyme B;
HSPA1B, heat shock protein 70 kDa 1B;
IL-1α, interleukin 1α;
IL-8, interleukin 8;
CCL3, C-C chemokine ligand 3;
CCL4, C-C chemokine ligand 4;
MMP-8, matrix metalloproteinase 8

### Example 3

### Biomarker-Based Risk Prediction Model

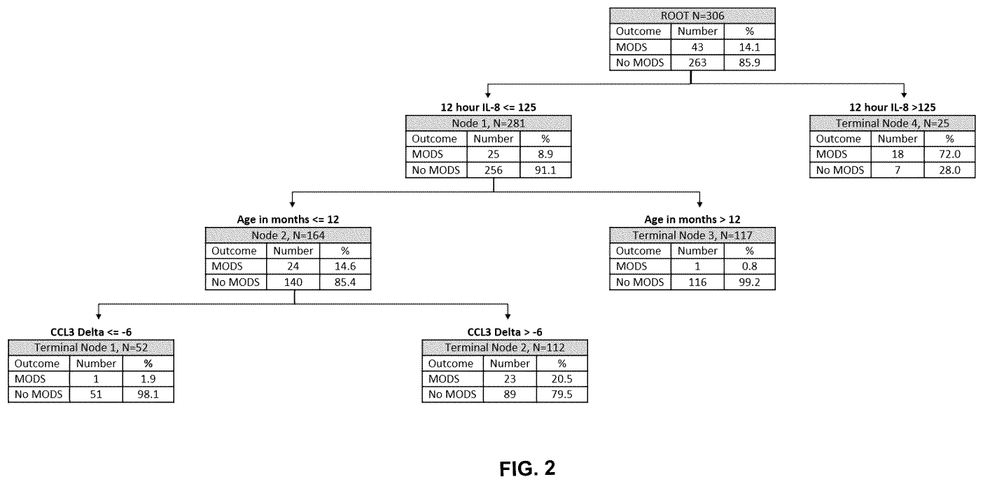

The newly derived PERSEVERE-CPB risk prediction model is shown in FIG. 2. The classification tree consists of two biomarker-based decision rules and one clinically based decision rule, namely IL-8 concentration at 12 hours, the change in serum concentration of CCL3 from 4 to 12 hours, and infant age category (<12 months). Each node contains the total number of subjects meeting the biomarker concentration or clinically based decision rule criteria, the number of subjects with or without persistent multiple organ dysfunction syndrome (MODS) at postoperative day (POD) 5, and the percentage of each respective outcome.

There were two low-risk terminal nodes (terminal nodes 1 and 3) in which subjects had <2% risk of developing persistent MODS. There was one intermediate-risk node with 23 patients (20.5%) who developed persistent organ dysfunction (terminal node 2). There was one high-risk node with persistent organ dysfunction in 72% of patients (terminal node 4). The area under the curve (AUC) for this tree was 0.86, with cross-validated estimate for AUROC of 0.75.

PERSEVERE-CPB performed well at determining risk of persistent MODS with model characteristics, as shown in Table 5. IL-8 concentration at 12 hours functioned as the upper tier decision rule, thus having the most predictive weight. Age less than 12 months was the second most important predictive variable, followed by change in the serum concentration of CCL3 from 4 to 12 hours.

TABLE 5
Diagnostic test characteristics of PERSEVERE-CPB.

| Number of subjects | 306 | |
|---|---|---|
| Number of True Positives | 41 | |
| Number of True Negatives | 167 | |
| Number of False Positives | 96 | |
| Number of False Negatives | 2 | |
| Sensitivity | 95% | (83; 99) |
| Specificity | 64% | (57; 69) |
| Positive Predictive Value | 30% | (23; 38) |
| Negative Predictive Value | 99% | (95; 100) |
| +Likelihood Ratio | 2.6 | (2.2; 3.1) |
| −Likelihood Ratio | 0.07 | (0.02; 0.28) |
| AUC | 0.86 | (0.81; 0.91) |
| Cross Validation AUC | 0.75 | (0.68; 0.84) |

Numbers in parenthesis represent 95% confidence intervals.
AUC: area under the curve;
+ likelihood ratio: positive likelihood ratio;
− likelihood ratio: negative likelihood ratio

### Example 4

### Prediction Performance

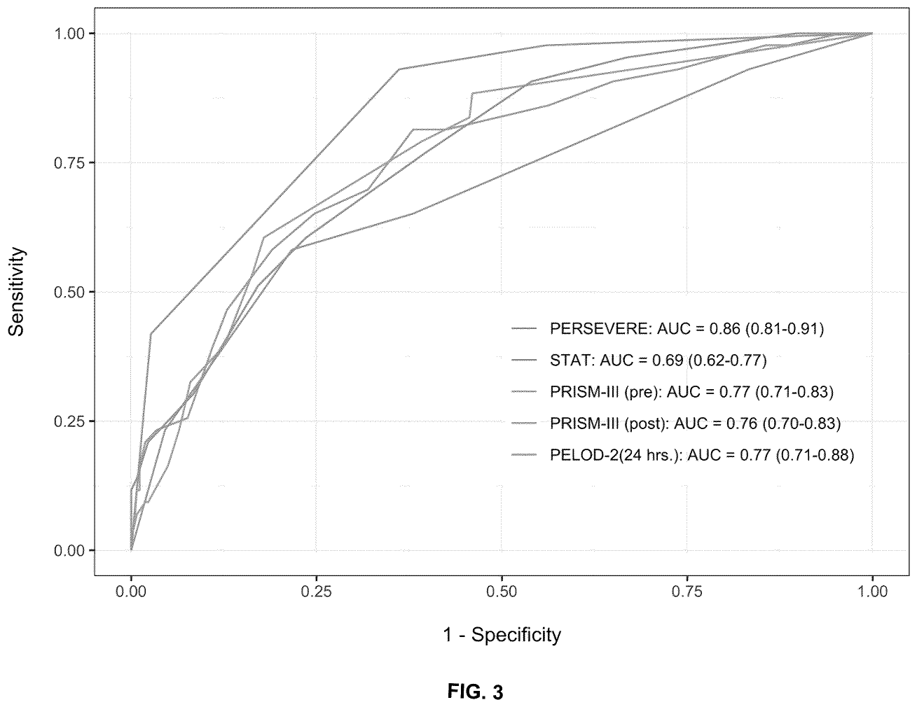

PERSEVERE-CPB had excellent performance for prediction of MODS: AUROC, 0.86 (95% CI 0.81; 0.91), as shown in FIG. 3. After cross validation, the PERSEVERE-CPB model's corrected AUROC 0.75 (95% CI of 0.68-0.84) still had good performance. PERSEVERE-CPB performed favorably to other validated risk scoring systems for prediction of MODS in the study cohort: STAT, 0.69 (0.62; 0.77); preoperative PRISM III, 0.77 (0.71; 0.83); and postoperative PRISM III, 0.76 (0.70; 0.83). PELOD-2 calculated using data from the first 24 hours after CPB had an AUROC of 0.77 (0.71; 0.88).

### Example 5

### Assessment of Postoperative Steroid Use and Outcome by PERSEVERE Risk Category

The portion of the cohort falling into the high-risk PERSEVERE-CPB category (terminal node 4 of the model) were more likely to receive steroids for post-operative hypotension as compared to those falling into the intermediate- and low-risk categories (35%, 22%, 2%, respectively; p<0.001). The high-risk cohort also experienced longer duration of ventilator and vasoactive support, longer CICU and hospital stays, and had higher in-hospital mortality compared to those falling into the intermediate- and low-risk categories, as shown in Table 6.

TABLE 6
Clinical outcomes by PERSEVERE-CPB Risk Strata

| | High Risk | | Intermediate Risk | | Low Risk | | p value |
|---|---|---|---|---|---|---|---|
| Number receiving | 14 | (56.0%) | 43 | (25.6%) | 13 | (7.7%) | <0.001 |
| steroids | | | | | | | |
| postoperative (%) | | | | | | | |
| Number receiving | 7 | (35.0%) | 15 | (22.4%) | 5 | (2.3%) | <0.001 |
| steroids for | | | | | | | |
| hypotension | | | | | | | |
| postoperative (%) | | | | | | | |
| Ventilator-free days | 21 | (9, 25) | 26 | (24, 28) | 28 | (27, 28) | <0.001 |
| Vasoactive-free days | 20 | (8, 23) | 25 | (23, 27) | 27 | (26, 27) | <0.001 |
| Number of in- | 5 | (20.0%) | 2 | (1.8%) | 0 | (0.0%) | <0.001 |
| hospital mortality | | | | | | | |
| (%) | | | | | | | |
| Number alive and | 12 | (48.0%) | 91 | (81.3%) | 164 | (97.1%) | <0.001 |
| out of the hospital | | | | | | | |
| by POD 28 (%) | | | | | | | |
| CICU LOS | 15 | (11, 30) | 5 | (2, 11) | 2 | (1, 4) | <0.001 |
| Hospital LOS | 25 | (20, 57) | 10 | (6, 19) | 6 | (4, 9) | <0.001 |

PERSEVERE-CPB risk category is based on terminal risk nodes from PERSEVERE-CPB model: Terminal node 4: high-risk; Terminal node 2: intermediate-risk; Terminal node 1 and 3: low-risk.
All data is presented as median (interquartile range) unless specified;
ventilator-free days: total days not receiving positive pressure ventilation out of 28 days;
vasoactive-free days: total days not requiring vasoactive or inotropic medications out of 28 days;
POD: postoperative day;
CICU: cardiac intensive care unit;
LOS: length of stay

### Example 6

### Biomarker Concentrations in Patients Receiving Dialysis

Dialysis, either continuous renal replacement therapy (CRRT) or PD, was used in 12 patients in the first 24 hours after separation from CPB, with 9 being infants. Peritoneal dialysis catheters drained ascites without active dialysis in the remaining 34 neonates.

Use of dialysis was associated with increased IL-8 at both 4 and 12 hours post-CPB. CCL-3 concentrations were higher in the dialysis group, but only 12 hour concentrations in the entire cohort were significant, as shown in Table 7.

TABLE 7
Interleukin-8 and chemokine ligand 3 concentrations in
patients receiving dialysis within 24 hours of surgery

| | | |
|---|---|---|
| Dialysis | No dialysis | p-value |

Entire Cohort (n = 306)

| IL-8 concentration (pg/mL) | | | | | | |
|---|---|---|---|---|---|---|
| 4 | hours | 245.7 | (105.3; 354.2) | 49.4 | (23.9; 94.3) | <0.001 |
| 12 | hours | 140.1 | (73.1; 313.4) | 35.4 | (15.9; 61.9) | <0.001 |
| CCL-3 concentration (pg/mL) | | | | | | |
| 4 | hours | 37.8 | (29.6; 58.3) | 22.9 | (13.1; 41.8) | 0.06 |
| 12 | hours | 61.4 | (43.7; 76.8) | 22.5 | (12.9; 35.1) | 0.001 |

Neonates only (n = 43)

| IL-8 concentration (pg/mL) | | | | | | |
|---|---|---|---|---|---|---|
| 4 | hours | 343.8 | (185.6; 374.9) | 98.0 | (56.3; 168.1) | 0.004 |
| 12 | hours | 179.1 | (102.3; 341.9) | 71.9 | (41.2; 99.2) | 0.002 |
| CCL-3 concentration (pg/mL) | | | | | | |
| 4 | hours | 37.1 | (31.0; 65.8) | 32.7 | (14.5; 58.4) | 0.366 |
| 12 | hours | 67.8 | (46.9; 76.5) | 31.1 | (22.5; 66.5) | 0.101 |

All data presented as median (interquartile range).
IL-8: interleukin-8;
CCL-3: C-C chemokine ligand 3

The various methods and techniques described above provide a number of ways to carry out the disclosure. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the disclosure extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the disclosure. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

1. Warren O J, Smith A J, Alexiou C, et al. The inflammatory response to cardiopulmonary bypass: part 1—mechanisms of pathogenesis. J Card Vasc Anesth 2009; 23:223-231.
2. Dybdahl B, Wahba A, Lien E. et al. The inflammatory response after open heart surgery release of heat-shock protein 70 and signaling through toll-like receptor 4. Circ 2002; 105:685-690.
3. Stocker C F, Shekerdemian L S, Visvanathan K, et al. Cardiopulmonary bypass elicits a prominent innate immune response in children with congenital heart disease. J Thorac Cardiovasc Surg 2004; 127:1523-1525.
4. Stocker C F, Shekerdemian L S, Horton S B, et al. The influence of bypass temperature on the systemic inflammatory response and organ injury after pediatric open surgery: a randomized trial. J Thorac Cardiovasc Surg 2011; 142:174-180.
5. Mahle W T, Matthews E, Kanter K R, et al. Inflammatory response after neonatal cardiac surgery and its relationship to clinical outcomes. Ann Thorac Surg 2014; 97:950-956.
6. Allan C K, Newburger J W, McGrath E, et al. The relationship between inflammatory activation and clinical outcome after infant cardiopulmonary bypass. Anesth Analg 2010; 111:1244-1251.
7. Seghaye M C. The clinical implications of the systemic inflammatory reaction related to cardiac operations in children. Cardiol Young 2003; 13:228-239.
8. Kubicki R, Grohmann J, Siepe M, et al. Early prediction of capillary leak syndrome in infants after cardiopulmonary bypass. Eur J Cardiothorac Surg 2013; 44:275-281.
9. Wheeler D S, Jeffries H E, Zimmerman J J, et al. Sepsis in the pediatric cardiac intensive care unit. World J Pediatr Congenit Heart Surg 2011; 2:393-39912.
10. Watson R S, Crow S S, Hartmann M E, et al. Epidemiology and outcomes of pediatric multiple organ dysfunction syndrome. Pediatr Crit Care Med 2017; 18(3): S4-S16.
11. Typpo K V, Lacroix J R. Monitoring severity of multiple organ dysfunction syndrome: new and progressive multiple organ dysfunction syndrome, scoring systems. Pediatr Crit Care Med 2017; 18(3)S17-S23.
12. Wong H R, Cvijanovich N, Allen G L, et al. Genomic expression profiling across the pediatric systemic inflammatory response syndrome, sepsis, and septic shock spectrum. Crit Care Med 2009; 37:1558-1566.
13. Standage S W, Wong H R. Biomarkers for pediatric sepsis and septic shock. Expert Rev Anti Infect Ther 2011; 9:71-79.
14. Kaplan J M, Wong H R. Biomarker discovery and development in pediatric critical care medicine. Pediatr Crit Care Med 2011; 12:165-173.
15. Wong H R, Salsibury S, Qiang Xiao, et al. The pediatric sepsis biomarker risk model. Critical Care 2012; 16:R147-183.
16. Wong H R, Caldwell J T, Cvijanovich N Z, et al. Prospective clinical testing and experimental validation of the Pediatric Sepsis Biomarker Risk Model. Sci Transl Med 2019; 11(518): eaax9000. doi: 10.1126/scitranslmed.aax9000. PMID: 31723040; PMCID: PMC7720682.
17. Wong H R. Genetics and genomics in pediatric septic shock. Crit Care Med 2012; 40:1618-1626
18. Wong H R, Weiss S L, Giuliano J S Jr, et al. Testing the prognostic accuracy of the updated pediatric sepsis biomarker risk model. PLoS ONE 2014; 9: e86242. doi:10.1371/j ournal.pone.0086242
19. Wong H R, Cvijanovich N Z, Anas N, et al. Developing a clinically feasible personalized medicine approach to pediatric septic shock. AJRCCM Articles in Press 2014; doi:10.1164/rccm.201410-18640C
20. Wong H R, Lindsell C J, Pettila V, et al. A multibiomarker-based outcome risk stratification model for adult septic shock. Crit Care Med 2014; 42:781-789.
21. Hayashi Y, Sawa Y, Fukuyama N, et al. Preoperative glutamine administration induces heat-shock protein 70 expression and attenuates cardiopulmonary bypass-induced inflammatory response by regulating nitric oxide synthase activity. Circ 2002; 106(20):260-267.
22. Beer L, Warszawska J M, Schenk P, et al. Intraoperative ventilation strategy during cardiopulmonary bypass attenuates the release of matrix metalloproteinases and improves oxygenation. J Surg Research 2015; 195(1): 294-302.
23. Beer L, Szerafin T, Mitterbauer A, et al. Low tidal volume ventilation during cardiopulmonary bypass reduces postoperative chemokine serum concentrations. Thorac Cardiovasc Surg 2014. 62(8):677-682.
24. Khabar K S A, ElBarbary M A, Khouqeer F, et al. Circulating endotoxin and cytokines after cardiopulmonary bypass: differential correlation with duration of bypass and systemic inflammatory response/multiple organ dysfunction syndromes. Clinical Immunology and Immunopathology 1997; 85(1): 97-103.
25. Madhok A B, Ojamaa K, Haridas V, et al. Cytokine response in children undergoing surgery for congenital heart disease. Pediatri Cardiol 2006; 27:408-413.
26. Carmona F, Manso P H, Vincente W V A, et al. Risk stratification in neonates and infants submitted to cardiac surgery with cardiopulmonary bypass: a multimarker approach combining inflammatory mediators, N-terminal pro-B-type natriuretic peptide and troponin I. Cytokine 2008; 42(3):317-324.
27. deFontnouvelle C A, Greenberg J H, Thiessen-Philbrook H R, et al. Interleukin-8 and tumor necrosis factor predict acute kidney injury after pediatric surgery. Ann Thorac Surg 2017; 104:2072-2079.
28. O'Brien S M, Clarke D R, Jacobs J P, et al. An empirically based tool for analyzing mortality associated with congenital heart disease. J Thorac Cardiovasc Surg 2009; 138(5): 1139-1153.
29. Jacobs J P, O'Brien S M, Pasquali S K, et al. Variation in outcomes for risk-stratified pediatric cardiac surgical operations: as analysis of the STS Congenital Heart Surgery Database. Ann Thorac Surg 2012; 94(2):564-571.
30. Gemke R J, van Vught J. Scoring systems in pediatric intensive care: PRISM III versus PIM. Intensive Care Med 2002; 28:204-207.
31. Goldstein B, Giroir B, Randolph A. International pediatric sepsis consensus conference: definitions for sepsis and organ dysfunction in pediatrics. Pediatr Crit Care Med 2005; 6:2-8.
32. Russell R A, Ghanayem N S, Kuhn E M, et al. Relationship between risk-adjustment tools and the pediatric logistic organ dysfunction score. World J Pediatr Congenit Heart Surg 2014; 15:16-21
33. Leteurtre S, Duhamel A, Salleron J, et al. PELOD-2: an update of the Pediatric logistic organ dysfunction score. Crit Care Med 2013; 41:1761-1773.
34. Umsted T M, Lu C K, Freeman W M, et al. The kinetics of cardiopulmonary bypass: a dual-platform proteomics study of plasma biomarkers in pediatric patients undergoing cardiopulmonary bypass. Artificial Organs 2012; 36(1):E1-E20.
35. Muller R, Mockel M. Logistic regression and CART in the analysis of multimarker studies. Clinica Chimica Acta 2008; 394_1-6.
36. Brown K L, Ridout D, Pagel C, et al. Incidence and risk factors for important early morbidities associated with pediatric cardiac surgery in a U K population. J Thorac Cardiovasc Surg 2019; 158(4):1185-1196.e736.
37. Brown K L, Ridout D A, Goldman A P, et al. Risk factors for long intensive care unit stay after cardiopulmonary bypass in children. Crit Care Med 2003; 31:28-33.
38. Gaies M G, Gurney J G, Yen A H, et al. Vasoactive-inotropic score as a predictor of morbidity and mortality in infants after cardiopulmonary bypass. Pediatr Crit Care Med 2010; 11(2):234-238.37.
39. Gaies M G, Jeffries H E, Niebler R A, et al. Vasoactive-inotropic score is associated with outcome after infant cardiact surgery: an analysis from the Pediatric Cardiac Critical Care Consortium and Virtual PICU System registries. Pediatr Crit Care Med 2014; 15(6):529-537.
40. Gessler P, Pfenninger J, Pfammatter J P, et al. Plasma levels of interleukin-8 and expression of interleukin-8 receptors on circulating neutrophils and monocytes after cardiopulmonary bypass in children. J Thorac Cardiovasc Surg 2003; 126(3):718-725.
41. Drennan S E, Burge K Y, Szyld E G, et al. Clinical and laboratory predictors for the development of low cardiac output syndrome in infants undergoing cardiopulmonary bypass: A pilot study. J Clin Med 2021; 10(4)712.
42. Gorjipour F, Totonchi Z, Dehaki M G, et al. Serum levels of interleukin-6, interleukin-8, interleukin-10 and tumor necrosis factor-□, renal function biochemical parameters and clinical outcomes in pediatric cardiopulmonary bypass surgery. Perfusion 2019; 34(8):651-659.
43. Liu K D, Altmann C, Smits G, et al. Serum interleukin-6 and interleukin-8 are early predictors of acute kidney injury and predict prolonged mechanical ventilation in children undergoing cardiac surgery: a case-control study. Crit Care 2009; 13(4)R104.
44. Rothenburger M, Tjan T D T, Schneider M, et al. The impact of the pro- and anti-inflammatory immune response on ventilation time after cardiac surgery. Cytometry B Clin Cytom 2003; 53(1):70-74.
45. Bhavasar I, Miller C S, Al-Sabbagh M. Macrophage inflammatory protein-1 alpha (MIP-1 alpha)/CCL3: as a biomarker. General Methods in Biomarker Research and their Applications 2015. 223-249.
46. Wolpe S D, Davatelis G, Sherry B, et al. Macrophages secrete a novel heparin-binding protein with inflammatory and neutrophil chemokinetic properties. J Exp Med 1988; 167(2):570-581
47. Wong H R, Cvijanovich N Z, Lindsell C J, et al. PERSEVERE II: Redefining the pediatric sepsis biomarker risk model with septic shock phenotype. Crit Care Med 2016; 44(11):2010-2017.
48. Miyata H, Murakami A, Tomotaki A, et al. Predictors of 90-day mortality after congenital heart surgery: the first report of risk models from a Japanese database. J Thorac Cardiovasc Surg 2014; 148:2201-2206.
49. Hill K D, Kannankeril P J, Jacobs J P, et al., for the STRESS Network Investigators. Methylprednisolone for heart surgery in infants—A randomized, controlled trial. N Engl J Med 2022; 387:2138-2149.
50. Sasser W C, Dabal R J, Askenazi D J, et al. Prophylactic peritoneal dialysis following cardiopulmonary bypass in children is associated with decreased inflammation and improved clinical outcomes. Congenit Heart Dis 2014; 9(2): 106-115.

1. A method of classifying a patient following cardiopulmonary bypass (CPB) as high risk of persistent multiple organ dysfunction syndrome (MODS), or other than high risk of persistent MODS, the method comprising:
obtaining a sample from a pediatric patient at about 12 hours post-CPB;
analyzing the 12 hours post-CPB sample to determine expression levels of one or more biomarkers comprising IL-8;
determining whether the expression level of IL-8 at 12 hours is greater than a respective cut-off IL-8 expression level; and
classifying the patient as high risk of persistent MODS, or other than high risk of persistent MODS, based on the determination of whether the expression level of IL-8 at 12 hours is greater than the respective cut-off IL-8 expression level.

2. The method of claim 1, further comprising:
determining whether the patient age is greater than 12 months; and
classifying the patient as high risk of persistent MODS, or other than high risk of persistent MODS, based on the determination of whether the expression level of IL-8 at 12 hours is greater than the respective cut-off IL-8 expression level, and whether the patient age is greater than 12 months.

3. The method of claim 1, further comprising:
obtaining a sample from a pediatric patient at about 4 hours post-CPB;
analyzing the 4 hours post-CPB sample to determine expression levels of one or more biomarkers comprising CCL3;
analyzing the 12 hours post-CPB sample to determine expression levels of one or more biomarkers comprising CCL3;
determining whether the change in expression level of CCL-3 from 4 to 12 hours is greater than a respective cut-off delta; and
classifying the patient as high risk of persistent MODS, or other than high risk of persistent MODS, based on the determination of whether the expression level of IL-8 at 12 hours is greater than the respective cut-off IL-8 expression level, whether the change in expression level of CCL-3 from 4 to 12 hours is greater than a respective cut-off delta, and whether the patient age is greater than 12 months.

4. The method of claim 2, wherein a classification of high risk of persistent MODS comprises:
a) an elevated level of IL-8;
and wherein a classification of other than high risk of persistent MODS comprises:
b) a non-elevated level of IL-8, and a patient age greater than 12 months; or
c) a non-elevated level of IL-8, and a patient age of less than or equal to 12 months.

5. (canceled)

6. The method of claim 3, wherein a classification other than high risk comprises a classification of low risk or intermediate risk, and wherein a classification of intermediate risk of persistent MODS comprises:
a non-elevated level of IL-8, a patient age of less than or equal to 12 months, and a non-elevated CCL3 delta;
and wherein a classification of low risk of persistent MODS comprises:
a non-elevated level of IL-8, and a patient age of less than or equal to 12 months, and an elevated CCL3 delta; or
a non-elevated level of IL-8, and a patient age greater than 12 months.

7. The method of claim 1, wherein the determined biomarker expression levels comprise expression levels of IL-8 and CCL3, and wherein biomarker expression levels are determined by quantification of serum protein biomarker concentrations, or wherein biomarker expression levels are determined by concentrations and/or by cycle threshold (CT) values.

8. (canceled)

9. (canceled)

10. The method of claim 7, wherein biomarker levels are determined by serum protein biomarker concentration, and wherein:
a) an elevated level of IL-8 corresponds to a serum IL-8 concentration greater than 125 pg/ml; and
b) an elevated CCL3 delta corresponds to a CCL3 delta greater than −6 pg/ml.

11. The method of claim 1, wherein the determination of whether the levels of the at least two biomarkers are non-elevated above a cut-off level comprises applying the biomarker expression level data to a decision tree comprising the two or more biomarkers.

12. (canceled)

13. The method of claim 1, wherein persistent MODS comprises cardiovascular, respiratory, renal, hepatic, hematologic, and/or neurologic dysfunction, and/or systemic inflammation, and/or increase in days requiring mechanical ventilatory support and cardiovascular support (e.g. use of vasoactive-inotropic infusion).

14. (canceled)

15. (canceled)

16. The method of claim 1, wherein the classification is combined with one or more patient demographic data and/or clinical characteristics and/or results from other tests or indicia of organ dysfunction and/or one or more additional biomarkers and/or platelet count, and/or wherein the classification is combined with one or more additional population-based risk scores.

17. The method of claim 16, wherein the one or more additional biomarkers is selected from the group consisting of: heat shock protein 70 kDa 1B (HSP70, HSPA1B), C—C Chemokine ligand 4 (CCL4), Granzyme B (GZMB), Interleukin-1α (IL-1α), Matrix metallopeptidase 8 (MMP8), Angiopoietin-1 (Angpt-1), Inter-Cellular Adhesion Molecule-1 (ICAM-1), Vascular cell adhesion molecule-1 (VCAM-1), P-selectin, E-selectin, and Platelet and endothelial cell adhesion molecule-1 (PECAM-1); and/or wherein the patient demographic data and/or clinical characteristics and/or results from other tests or indicia of organ dysfunction comprise at least one selected from the group consisting of: the presence or absence or chronic disease, and/or the gender, race, ethnicity, and/or co-morbidities of the patient, and/or wherein the one or more population-based risk scores comprises at least one selected from the group consisting of: Pediatric Sepsis Biomarker Risk Model (PERSEVERE), Pediatric Sepsis Biomarker Risk Model II (PERSEVERE II), Pediatric Risk of Mortality (PRISM), PRISM III, Pediatric Index of Mortality (PIM), and Pediatric Logistic Organ Dysfunction (PELOD).

18. (canceled)

19. (canceled)

20. (canceled)

21. (canceled)

22. (canceled)

23. The method of claim 1, further comprising administering a treatment comprising one or more high risk therapy to a patient that is classified as high risk, or administering a treatment excluding a high risk therapy to a patient that is not high risk, or to provide a method of treating a pediatric patient following CPB.

24. The method of claim 23, wherein the one or more high risk therapy comprises at least one selected from the group consisting of: biological and/or immune enhancing therapy, extracorporeal membrane oxygenation/life support, plasmapheresis, peritoneal dialysis, pulmonary artery catheterization, high volume continuous hemofiltration, steroids, adjuvant hemoperfusion, and/or plasma filtration and/or adsorption therapies.

25. (canceled)

26. The method of claim 1, wherein the patient is enrolled in a clinical trial.

27. The method of claim 26, wherein the patient is classified as high risk, and wherein the method comprises prognostic enrichment through enrollment of the high risk patient in the clinical trial, and further comprising administering a treatment comprising one or more high risk therapy to the patient in the clinical trial.

28. (canceled)

29. (canceled)

30. The method of claim 1, wherein the risk of persistent MODS comprises a risk of developing persistent MODS by day 5 following CPB.

31. The method of claim 1, comprising improving an outcome in a pediatric patient following CPB.

32. The method of claim 1, as part of a companion diagnostic or a point of care device or kit.

33. A diagnostic kit, test, or array comprising a reporter hybridization probe, and a capture hybridization probe specific for each of two or more mRNA, DNA, or protein biomarkers selected from the group consisting of: IL-8 and CCL3.

34. The diagnostic kit, test, or array of claim 33, wherein the biomarkers further comprise one or more of heat shock protein 70 kDa 1B (HSP70, HSPA1B), C—C Chemokine ligand 4 (CCL4), Granzyme B (GZMB), Interleukin-1α (IL-1α), and/or Matrix metallopeptidase 8 (MMP8).

35.-40. (canceled)
